(19) United States
(12) Patent Application Publication
(10) Pub. No.: US 2010/0121396 A1
(43) Pub. Date: May 13, 2010

(54) ENHANCED HEMODYNAMICS THROUGH ENERGY-EFFICIENT ANODAL PACING

(75) Inventors: Jong Gill, Valencia, CA (US); Gene A. Bornzin, Simi Valley, CA (US)

Correspondence Address: PACESETTER, INC., 15900 VALLEY VIEW COURT, SYLMAR, CA, 91392-9221, US

(73) Assignee: PACESETTER, INC., Sylmar, CA (US)

(21) Appl. No.: 12/267,941

(22) Filed: Nov. 10, 2008

(51) Int. Cl. A61N 1/365

(52) U.S. Cl. 607/17

(57) ABSTRACT

An implantable device may employ anodal-based cardiac stimulation to improve hemodynamics. Anodal pacing may be provided on a conditional basis (e.g., upon detection of a defined condition). An implantable device may provide anodal pacing or cathodal pacing according to a defined ratio. An implantable device may use automatic capture detection to determine a pacing energy level that provides effective anodal pacing while attempting to minimize the power consumption associated with the anodal pacing.

### CROSS-REFERENCE TO RELATED APPLICATIONS

This application contains subject matter that is related to copending U.S. patent application Ser. No. 11/961,720, filed Dec. 20, 2007, titled “Method and Apparatus with Anodal Capture Monitoring.”

### TECHNICAL FIELD

This application relates generally to implantable cardiac stimulation devices and more specifically to anodal pacing, also referred to as anodal stimulation.

### BACKGROUND

An implantable medical device, such as an implantable cardiac rhythm management device (e.g., a pacemaker, a defibrillator, or a cardioverter), may be used to monitor cardiac function and provide therapy for a patient who suffers from cardiac arrhythmia. For example, in an attempt to maintain regular cardiac rhythm, an implantable device may track the type and timing of native cardiac signals. In this way, the implantable device may determine whether cardiac events (e.g., contractions) are occurring and whether they are occurring at the proper times. In the event contractions are not occurring or are occurring at undesirable times, the implantable device may stimulate the heart in an attempt to restore proper cardiac rhythm. For example, an implantable device may stimulate the cardiac muscles of one or more chambers of the heart by delivering electrical pulses via one or more leads implanted in or near the chamber(s).

The implantable device also may track cardiac signals through the use of these implanted leads. For example, the implantable device may process signals received via the leads and then attempt to characterize the received signals as a particular cardiac event. Such cardiac events may include, for example, P-waves, R-waves, and T-waves. A P-wave corresponds to a contraction (depolarization) of an atrium. A QRS complex (comprising an R-wave) corresponds to a contraction (depolarization) of a ventricle. A T-wave corresponds to a return to a resting state (repolarization) of a ventricle.

By analyzing the type and timing of these cardiac events, the implantable device may determine whether therapy should be provided and, if so, the type of therapy to be provided (e.g., stimulation pulses). For example, if the implantable device detects cardiac events at the appropriate relative times, the device may simply continue monitoring the event. In contrast, if a particular cardiac event has not been detected for a defined period of time, the implantable device may deliver an appropriate stimulation (e.g., pacing) pulse to the heart. If too many cardiac events of a given type are received over a defined time period (e.g., a tachycardia condition is detected), the implantable device may provide a different form of therapy.

In some aspects, an implantable device may be used in conjunction with one or more implantable leads that provide pacing and/or sensing via a unipolar electrode configuration or a bipolar electrode configuration. In a unipolar configuration, pacing stimulation pulses may be applied or cardiac signals may be sensed between a single electrode carried by the lead that is electrically coupled with a nearby heart chamber and a relatively distant electrode such as the case of the implantable device. During “cathodal stimulation”, i.e., “cathodal pacing”, the lead electrode that is coupled with the heart chamber may serve as the cathode (negative pole) and the distant electrode may serve as the anode (positive pole). Conversely, during “anodal stimulation”, i.e., “cathodal pacing”, the lead electrode that is coupled with the heart chamber may serve as the anode and the distant electrode may serve as the cathode. In a bipolar configuration, pacing stimulation pulses may be applied or cardiac signals may be sensed between a pair of relatively closely spaced electrodes carried by the lead, at least one of which is electrically coupled with a nearby heart chamber. In the case where only one electrode is coupled to the heart, the coupled electrode serves as the cathode during cathodal stimulation and the anode during anodal stimulation. In the case where both electrodes are coupled to the heart, either electrode may serve as the anode electrode with the other serving as the cathode electrode.

Conventionally, implantable devices employ cathodal stimulation as opposed to anodal stimulation since tissue capture may be achieved at lower stimulation voltages when cathodal stimulation is used. By achieving tissue capture at lower voltages, power consumption of an implantable device may be reduced, thereby increasing the life of the battery of the implantable device.

### SUMMARY

A summary of several sample aspects of the disclosure and embodiments of an apparatus constructed or a method practiced according to the teaching herein follows. It should be appreciated that this summary is provided for the convenience of the reader and does not wholly define the breadth of the disclosure. For convenience, one or more aspects or embodiments of the disclosure may be referred to herein simply as “some aspects” or “some embodiments.”

The disclosure relates in some aspects to anodal cardiac stimulation or pacing whereby a stimulation pulse of positive polarity is applied across an anode electrode coupled to a heart chamber and a cathode electrode that may or may not be coupled to a heart chamber. In some aspects, hemodynamics for a patient may be improved through the use of anodal cardiac stimulation. The disclosure also involves cathodal cardiac stimulation or pacing whereby a stimulation pulse of negative polarity is applied across a cathode electrode coupled to a heart chamber and an anode electrode that may or may not be coupled to a heart chamber.

The disclosure relates in some aspects to conditionally providing anodal stimulation. For example, an implantable device may be configured to use anodal stimulation rather than cathodal stimulation under certain circumstances. To this end, the implantable device may be configured to monitor cardiac-related conditions in a patient. For example, if it is determined that a heart failure or ischemia condition of a patient is worsening, anodal stimulation may be employed instead of cathodal stimulation.

The disclosure relates in some aspects to providing anodal stimulation or cathodal stimulation according to a defined ratio. For example, an implantable device may provide anodal stimulation a given percentage of the time (e.g., 75%) and provide cathodal stimulation the remainder of the time (e.g., 25%).

The disclosure relates in some aspects to determining a minimum pacing energy level that provides effective anodal stimulation. In some aspects, an automatic capture scheme is employed in conjunction with anodal stimulation. In some aspects, the pacing energy for providing anodal capture at each of a plurality of electrodes is determined based on current levels associated with capture thresholds determined by independently applying stimulation with each electrode.

### BRIEF DESCRIPTION OF THE DRAWINGS

These and other features, aspects, and advantages will be more fully understood when considered with respect to the following detailed description, the appended claims, and the accompanying drawings, wherein:

FIG. 1 is a simplified flowchart of an embodiment of operations that may be performed to provide anodal stimulation;

FIG. 2 is a simplified block diagram of an embodiment of components that may be employed to provide anodal stimulation;

FIG. 3 is a simplified flowchart of an embodiment of operations that may be performed to identify at least one electrode and associated stimulation energy for anodal stimulation;

FIG. 4 is a simplified flowchart of an embodiment of operations that may be performed to determine stimulation energy for anodal stimulation;

FIG. 5 is a simplified flowchart of an embodiment of operations that may be performed to provide anodal and cathodal stimulation according to a defined ratio;

FIG. 6 is a simplified flowchart of an embodiment of operations that may be performed to conditionally provide anodal stimulation;

FIG. 7 is a simplified diagram of an embodiment of an implantable stimulation device in electrical communication with one or more leads implanted in a patient's heart for sensing conditions in the patient, delivering therapy to the patient, or providing some combination thereof; and

FIG. 8 is a simplified functional block diagram of an embodiment of an implantable cardiac device, illustrating basic elements that may be configured to sense conditions in the patient, deliver therapy to the patient, or provide some combination thereof.

In accordance with common practice the various features illustrated in the drawings may not be drawn to scale. Accordingly, the dimensions of the various features may be arbitrarily expanded or reduced for clarity. In addition, some of the drawings may be simplified for clarity. Thus, the drawings may not depict all of the components of a given apparatus or method. Finally, like reference numerals may be used to denote like features throughout the specification and figures.

### DETAILED DESCRIPTION

The description that follows sets forth one or more illustrative embodiments. It should be appreciated that the teachings herein may be embodied in a wide variety of forms and that the specific structural and functional details disclosed herein are merely representative and do not wholly define the scope of the disclosure. For example, based on the teachings herein one skilled in the art should appreciate that one or more of the disclosed structural and functional details may be incorporated in an embodiment independently of any other structural or functional details. Thus, an apparatus may be implemented or a method practiced using any number of the structural or functional details set forth in any disclosed embodiment(s). Also, an apparatus may be implemented or a method practiced using other structural or functional details in addition to or other than the structural or functional details set forth in any disclosed embodiment(s).

The disclosure relates in some aspects to using anodal stimulation (e.g., cardiac pacing) to improve hemodynamics for a patient. For example, in some aspects anodal stimulation may improve cardiac conduction velocity and contractability for a patient. In some aspects, anodal stimulation may improve hemodynamics of heart failure patients when applied to the left ventricle during cardiac resynchronization therapy (“CRT”).

The disclosure also relates in some aspects to providing anodal stimulation while managing the associated energy consumption of an implanted device. For example, techniques are described for reducing energy consumption that may otherwise result from anodal stimulation. In this way, anodal stimulation may be effectively used while mitigating the negative impact this form of therapy has on battery longevity.

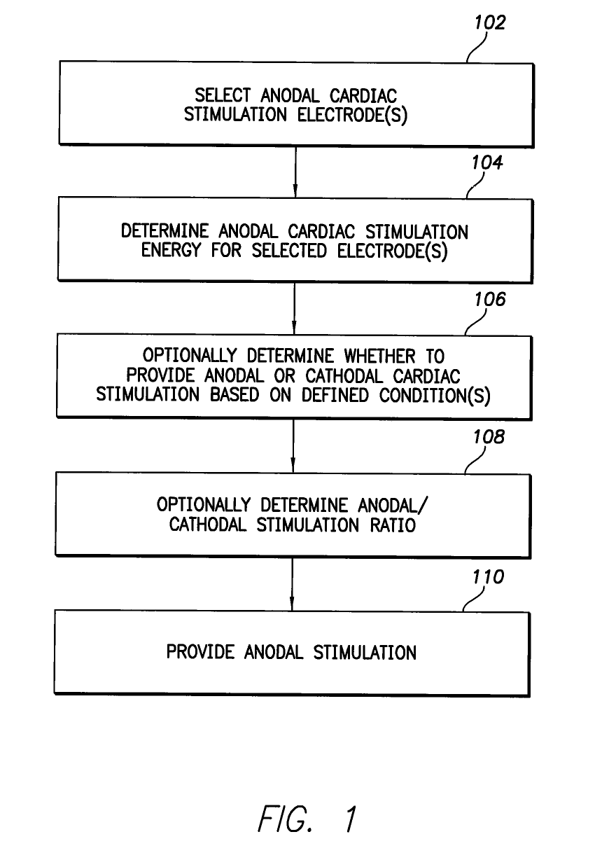

FIG. 1 provides an overview of several sample operations that may be performed in conjunction with providing anodal stimulation. In practice, different embodiments may employ one or more of the operations of FIG. 1.

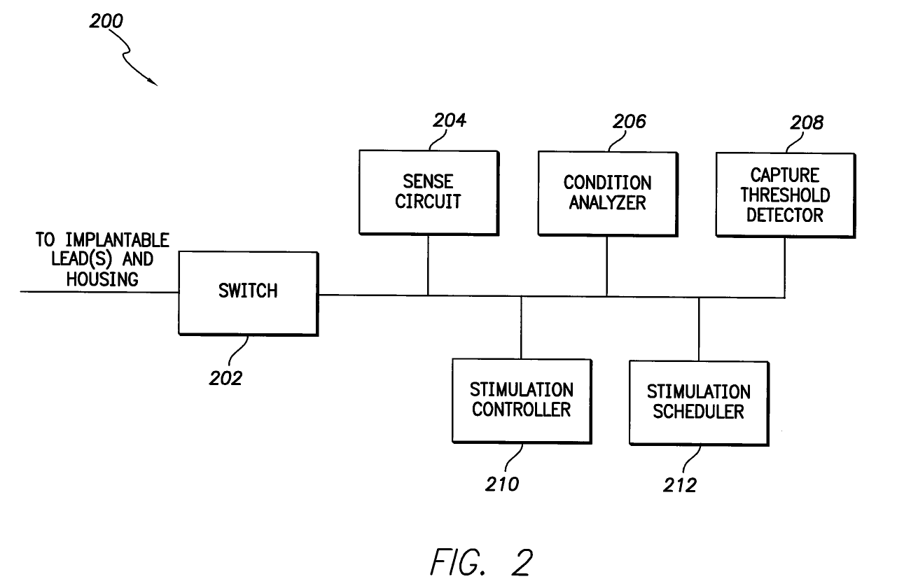

For convenience, the operations of FIG. 1 (or any other operations discussed or taught herein) may be described as being performed by specific components. For example, referring to FIG. 2, the operations described herein may be performed by an apparatus 200 (e.g., an implantable device) that includes one or more of the illustrated components. It should be appreciated, however, that the described operations may be performed by other types of components and may be performed using a different number of components. It also should be appreciated that one or more of the operations described herein may not be employed in a given implementation.

As represented by block 102 of FIG. 1, the apparatus 200 may be configured to select one or more electrodes (e.g., incorporated into one or more implantable leads) to be used for anodal cardiac stimulation. In some aspects, this may involve using a switch 202 to configure one or more electrodes (e.g., a tip electrode coupled to a heart) as an anode and configuring one or more other electrodes (e.g., a shocking coil or a housing of an implantable device that is not coupled to the heart) as a cathode for cardiac stimulation operations. In some embodiments, a defined set of electrodes may be used for anodal stimulation. For example, upon implant the apparatus 200 may be configured (e.g., corresponding settings are programmed) such that a given electrode is designated as an anode and another electrode is designated as a cathode for stimulation operations. In some embodiments the smallest electrode or electrodes in a set of electrodes may be selected as the anode. In some embodiments multi-site pacing may be employed whereby cardiac stimulations are provided in a manner that results in capture at each of multiple electrodes located at different locations in, on or near cardiac tissue.

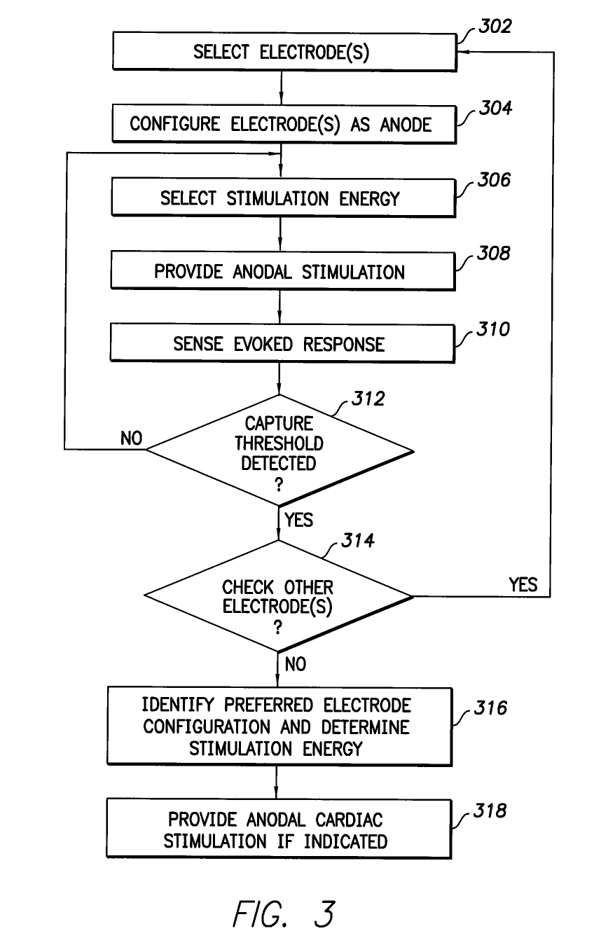

Alternatively, as will be described in more detail in conjunction with FIG. 3, in some embodiments the apparatus 200 may be configured to select one or more electrodes in an attempt to minimize the power consumed during anodal stimulation. For example, the operations of block 102 may involve identifying an electrode combination that uses the least amount of stimulation energy to achieve anodal capture. Here, during an electrode selection test, a sense circuit 204 may be coupled via the switch 202 to one or more electrodes to sense any evoked response signals that occur as a result of an anodal stimulation test pulse. A capture threshold detector 108 may be configured to process these sensed signals to determine whether tissue capture has occurred and to systematically adjust the anodal stimulation energy level to identify an anodal capture threshold for the current electrode configuration. These operations may then be performed for different electrode configurations to identify, for example, the electrode combination associated with the lowest stimulation energy level that results in capture.

As represented by block 104, the apparatus 200 also is configured to determine the cardiac stimulation energy to be used when applying anodal stimulation via the selected electrode or electrodes. For example, this energy level may be determined by adding a safety margin (e.g., 0.5 V) to a stimulation voltage level corresponding to the anodal capture threshold for the selected electrode(s) or by multiplying this stimulation voltage level by a safety factor (e.g., 1.25 or 1.5).

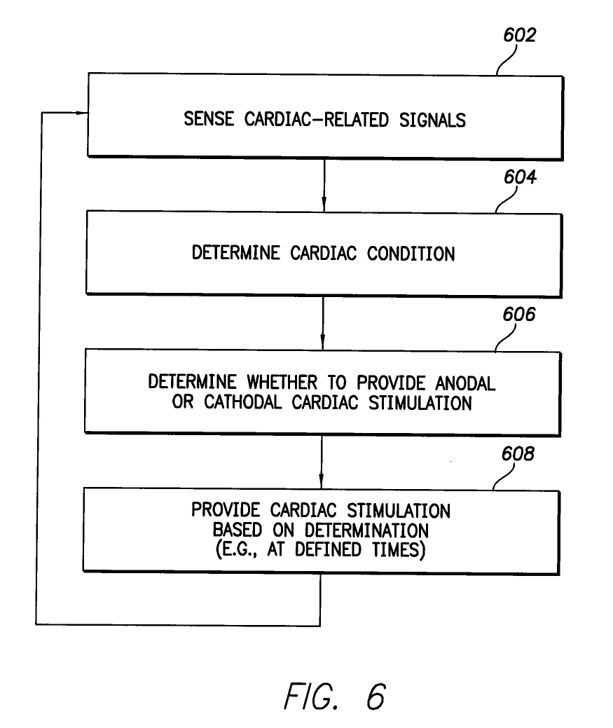

As represented by block 106, in some implementations the apparatus 200 may conditionally provide anodal cardiac stimulation. For example, rather than always provide anodal stimulation via a given set of electrodes whenever stimulation is indicated for the patient, the apparatus 200 may provide anodal stimulation if a certain condition is met or certain conditions are met. Conversely, if each condition is not met when stimulation is indicated for the patient, the apparatus 200 may be configured to provide cathodal stimulation. To this end, the apparatus 200 may include a condition analyzer 206 that is configured to monitor one or more conditions associated with a patient. These aspects of the disclosure are treated in more detail below in conjunction with FIG. 6.

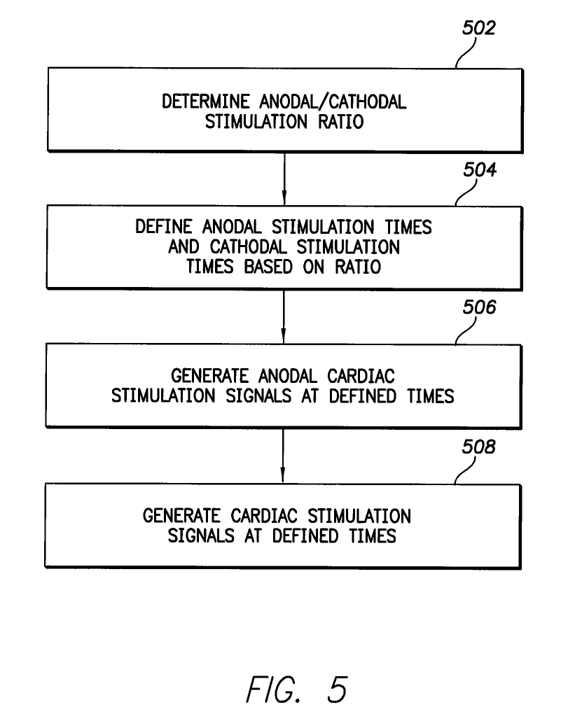

As represented by block 108, in some implementations the apparatus 200 may provide anodal and cathodal cardiac stimulation according to a defined anodal/cathodal stimulation ratio. For example, a stimulation scheduler 212 may schedule anodal cardiac stimulation 75% of the time that stimulation is indicated for a patient and schedule cathodal cardiac stimulation the remaining 25% of the time. These aspects of the disclosure are treated in more detail below in conjunction with FIG. 5.

As represented by block 110, the apparatus 200 (e.g., a stimulation controller 210) may provide anodal cardiac stimulation in accordance with one or more of the above operations. Here, the switch 202 may configure one or more electrodes on one or more implantable leads as an anode and one or more other electrodes as a cathode. This configuration may be based on, for example, the electrode selection described at block 102, based on some other form of dynamic (e.g., automatic) electrode selection procedure, or based on a defined (e.g., fixed) electrode configuration. The stimulation controller 210 may then cause an appropriate level of energy to be applied to the electrodes (e.g., as determined at block 104 or in some other manner). To this end, the stimulation generator 210 may include or operate in conjunction with one or more signal generators configured to provide suitable cardiac stimulation signals (e.g., pacing pulses).

Referring now to FIG. 3, various aspects of selecting an electrode configuration for anodal stimulation and determining stimulation energy for the electrode will be treated in more detail. In some aspects, the example of FIG. 3 relates to performing a capture detection test for different electrode combinations as mentioned above.

As represented by block 302, the switch 202 selects one or more electrodes for a given iteration of a capture threshold test. For example, for a scenario where anodal stimulation is to be applied to the left ventricle, in a first iteration a tip electrode of a lead implanted in the coronary sinus may be designated as an anode and a shocking coil (e.g., implanted in the right ventricle) may be designated as a cathode. For a subsequent iteration of the test, a ring electrode of the coronary sinus lead may be designated as an anode and the same shocking coil or a different electrode may be designated as a cathode. It should be appreciated that a wide variety of electrode combinations are possible. For example, a given electrode combination may include one or more of: a tip electrode, a ring electrode, a shocking coil, a housing of an implantable device, a pericardial electrode, or some other type of electrode. Here, one or more electrodes (that are coupled to a heart) may be designated as an anode and one or more electrodes (that are either coupled or not coupled to a heart) may be designated as a cathode. Also, an electrode may be configured in a unipolar configuration, a bipolar configuration, or some other configuration.

As represented by block 304, when a given electrode configuration is to be tested, the switch 202 configures the selected electrode(s) for anodal stimulation. For example, the switch 202 may couple a positive terminal of an energy source to each electrode designated as an anode and couple a negative terminal of an energy source to each electrode designated as a cathode.

Blocks 306-312 relate to identifying a capture threshold for a given electrode configuration. In some aspects these operations may be performed by cooperation of the capture threshold detector 208, the sense circuit 204, and the stimulation controller 210.

At block 306, the capture threshold detector 208 selects the stimulation energy (e.g., a voltage level to be applied to the pacing electrodes) to be used for a given iteration of the test. In a typical implementation, the capture test is commenced using a high energy level or a level at which capture is currently occurring. Conversely, in some implementations a capture test may be commenced at a low energy level or a level at which it is known that capture does not occur.

At block 308, the stimulation controller 210 may generate anodal stimulation signals using the currently designated energy level for one or more cardiac cycles when stimulation is indicated for the patient during the test. These stimulation signals are coupled via the switch 202 to the electrodes designated by the current electrode configuration. In accordance with conventional practice, stimulation may be indicated, for example, when certain intrinsic activity (e.g., an R-wave or a P-wave) is not detected within a designated detection window.

At block 310, the sense circuit 204 senses for any evoked response that occurs as a result of the anodal stimulation. Here, the sense circuit 204 may be coupled via the switch 202 to one or more implantable electrodes to sense cardiac activity.

At block 312, the capture threshold detector 208 analyzes the evoked response signals, if any, to determine whether the stimulation resulted in tissue capture. This analysis may involve various operations and may be implemented in various ways. For example, the capture threshold detector 208 may integrate the evoked response signal information sensed during a defined period of time (e.g., a detection window). If the resulting integral is greater than a threshold level, capture may be deemed to have occurred.

Provisions also may be made to distinguish “anodal capture”, i.e., an evoked response that occurs as a result of anodal stimulation, from “cathodal capture”, i.e., an evoked response that occurs as a result of cathodal stimulation. For example, in some electrode configurations, cathodal capture may occur instead of or in addition to anodal capture when a stimulation signal is applied to the patient's heart. In some implementations anodal capture may be distinguished from cathodal capture based on morphology differences between the evoked responses associated with the different types of capture. That is, the morphology of the evoked response that results from anodal capture by a given electrode may be different than the morphology of the evoked response that results from cathodal capture by another electrode.

In some implementations anodal capture may be distinguished from cathodal capture based on timing differences (e.g., a phase shift) between the evoked responses associated with the different types of capture. In some aspects, such a timing difference may be attributed to different electrical paths (e.g., different distances or different impedance paths) that exist between cardiac tissue and the respective anode and cathode electrodes. For example, an electrode that is situated on “good” cardiac tissue (e.g., cells that are not damaged in some way) may result in an “earlier” evoked response than an electrode that is in a blood pool (e.g., a ring electrode) or that is in contact with “bad” cardiac tissue.

The operations performed during subsequent iterations through the loop associated with blocks 306-312 will depend on whether the initial stimulation energy was a high level or a low level.

If the initial energy level was high (capture initially achieved), the test involves decreasing the energy level at block 306 and checking for capture at blocks 308-312. This process is repeated until capture is lost. The value at which capture last occurred before capture is lost may then be designated as the capture threshold.

If the initial energy level was low (capture not initially achieved), the test involves increasing the energy level at block 306 and checking for capture at blocks 308-312. This process is repeated until capture is achieved. The value at which capture is first achieved may then be designated as the capture threshold.

In either case, provisions may be made to ensure that the patient does not miss a prescribed cardiac stimulation pulse. For example, a backup stimulation pulse may be generated for those instances of the test where capture is not detected at blocks 310 and 312.

As represented by block 314, the capture threshold detection test may then be repeated for additional electrode configurations, if applicable. That is, other electrodes are selected at block 302 and configured in an anode/cathode relationship at block 304. Capture threshold is detected for this electrode configuration by performing the loop of blocks 306-312. Here, for each anode/cathode electrode configuration, the stimulation controller 210 maintains a record of the stimulation energy provided to the electrodes when the capture threshold is detected.

As represented by block 316, the stimulation controller 210 may then identify the electrode configuration and the stimulation energy to be used for normal anodal stimulation operations. In some embodiments, the electrode configuration that achieved capture at the lowest stimulation energy level may be selected as the preferred electrode configuration.

The stimulation energy to be used for anodal stimulation may be based on energy level associated with the capture threshold for the selected electrode configuration and some form of safety margin. For example, as discussed above a safety margin may be added to the energy level or the energy level may be multiplied by a safety factor.

As represented by the dashed line between blocks 316 and 318, the anodal stimulation calibration procedure concludes at this point and the apparatus 200 may return to a normal monitoring and stimulation mode, during which cathodal stimulation, anodal stimulation or a combination of both may be provided. As represented by block 318, if anodal stimulation in called for during normal monitoring and stimulation, the stimulation controller 210 may use the designated anodal-stimulation electrode configuration and energy level identified in block 316 to provide anodal cardiac stimulation.

The process of determining the desired stimulation energy level to be used may depend in some aspects on the particular configuration of the electrodes used for providing stimulation. For example, when a unipolar-type configuration is employed (e.g., where a shocking coil or the housing is the cathode), determining the stimulation energy may simply involve determining the amount of energy supplied to the unipolar anode electrode.

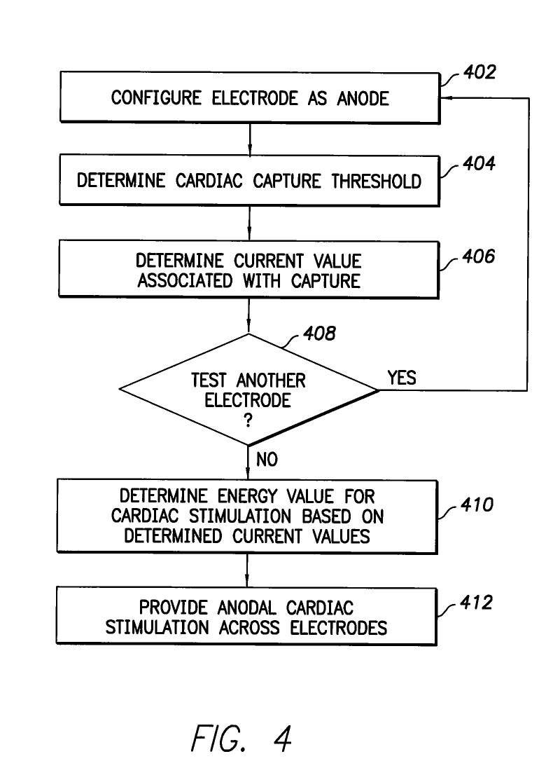

In contrast, when capture is intended to occur at each of a plurality of electrodes in an electrode set (e.g., where the electrodes are of a more comparable size such as in a bipolar-type configuration), the process of determining the desired stimulation energy level may be more complicated. For example, referring to FIG. 4, a method is described for determining the desired stimulation energy for stimulating tissue at each of a plurality of electrodes. In some aspects, this method involves determining energy levels (e.g., current values) associated with capture for each electrode independently, and then determining the desired stimulation energy based on these energy levels.

Blocks 402-408 relate to determining a current value associated with anodal capture for each electrode in a given set of electrodes. For example, the set of electrodes may include one electrode implanted in or near the left ventricle (e.g. in the coronary sinus) and another electrode implanted in the right ventricle. Alternatively, the set of electrodes may include several electrodes positioned within the coronary vascular over the left ventricle.

At block 402, the switch 202 configures one of the electrodes as an anode and another electrode as a cathode. For example, the electrode may be configured in a unipolar configuration where the cathode is a shocking coil or the housing. Alternatively, the cathode may be another one of the electrodes within the electrode set.

At block 404 the capture threshold detector 208 determines the anodal capture threshold for the electrode configured as the anode. This may involve, for example, operations similar to those discussed above at FIG. 3.

At block 406 the stimulation controller 210 determines a current value associated with the capture threshold. This may involve, for example, determining the amount of current that flows through the anode electrode when capture is just barely achieved. A current value such as this may be determined in various ways. For example, in some implementations this current value may be determined from the applied voltage and the impedance of the electrode circuit. To this end, the stimulation controller 210 may record the voltage level applied to the anode electrode that achieves the capture threshold. The stimulation controller 210 may then include or operate in conjunction with an impedance measurement circuit that measures the impedance of the electrode circuit.

As represented by block 408, the above operations are repeated for each electrode in the set. That is, a next electrode is configured as an anode at block 402, the capture threshold for this anode electrode is identified at block 404, and the corresponding current value is determined at block 406.

At block 410 the stimulation controller 210 determines the energy value to be used to effectuate capture at each electrode in the set of electrodes based on the current values determined at block 406. In some implementations this energy value may be based on the maximum current value of the current values identified at block 406 for the electrodes in the set. In this case, the stimulation controller 210 may determine the energy (e.g., voltage) level that will provide this level of current to the electrodes of the set. In addition, as discussed above the final energy level to be used for stimulation may be adjusted to provide a sufficient margin of safety.

As represented by the dashed line between blocks 410 and 412, the anodal stimulation energy determination procedure concludes at this point and the apparatus 200 may return to a normal monitoring and stimulation mode, during which cathodal stimulation, anodal stimulation or a combination of both may be provided. As represented by block 412, if multisite anodal capture is called for during normal monitoring and stimulation, the switch 202 configures the anode electrode set and the stimulation controller 210 uses the determined energy value of block 410 to provide the anodal cardiac stimulation signal sufficient to effectuate capture at each electrode in the anode electrode set.

Referring now to FIG. 5, in some embodiments the apparatus 200 may provide both anodal cardiac stimulation and cathodal cardiac stimulation. For example, one or more electrodes may be configured to provide anodal stimulation at certain times and one or more electrodes may be configured to provide cathodal stimulation at certain times. These times may or may not overlap. In addition, the stimulation may be provided using one or more common electrodes or different electrodes.

By mixing anodal and cathodal stimulation, the overall energy consumption of an implantable device may be reduced as compared to a case that uses anodal stimulation exclusively. The use of such mixing may, however, result in a reduction in the hemodynamic improvement that may otherwise be achieved by exclusive anodal stimulation.

As represented by block 502, in some aspects anodal cardiac stimulation and cathodal cardiac stimulation may be provided based on an anodal/cathodal stimulation ratio. This ratio may take various forms. For example, in some cases this ratio may define the percentage of time that anodal cardiac stimulation is to be used versus the percentage of time that cathodal cardiac stimulation is to be used. Such a ratio may be expressed, for example, as a number ratio (e.g., 3:1), as a percentage (e.g., 75% versus 25%), a period of time (e.g., 5 minutes versus 2 minutes), as a number of stimulation pulses (e.g., 100 pulses versus 30 pulses), or in some other suitable manner.

The ratio may be defined in various ways. For example, in some cases a default ratio may be programmed into an implantable device (e.g., during an implant procedure). In some cases different ratios may be used under different circumstances (e.g., depending on the condition of the patient or some other factor). In some cases a given ratio may be adapted (e.g., depending on the condition of the patient or some other factor).

As represented by blocks 504 and 506, the stimulation scheduler 212 defines anodal stimulation times and cathodal stimulation times based on the ratio. For example, the stimulation scheduler 212 may designate times at which anodal stimulation and cathodal stimulation are to be invoked or may designate when to switch from one form of stimulation to the other (e.g., after a defined number of stimulation pulses or at a certain time). To this end, the stimulation scheduler 212 may comprise a counter or a timer that tracks the amount of time or number of times that anodal stimulation has been invoked and cathodal stimulation has been invoked.

As represented by block 508, the stimulation controller 210 generates the cardiac stimulation signals at the designated times. Here, the stimulation controller 210 may control the switch 202 to configure electrodes in the appropriate manner at the designated times.

It should be appreciated that mixed anodal and cathodal stimulation may be implemented using various electrode configurations. For example, in some cases a unipolar anode configuration and a unipolar cathode configuration may be used. In some cases a bipolar electrode configuration may be used, where the polarities of the signals applied to the electrodes are switched when switching from anodal stimulation to cathodal stimulation, and vice versa.

In some embodiments, anodal stimulation may be provided on an on-demand basis. For example, in some implementations anodal stimulation may be provided only under conditions where the expected improvement in hemodynamics is particularly desirable. As a specific example, it may be desirable to achieve enhanced hemodynamic performance for a NYHA class III or class IV CRT patient. Conversely, if it is determined that the condition of the patient is acceptable or has improved, the additional hemodynamic improvement may not be as important. Hence, anodal stimulation may not be invoked or may be terminated (or reduced) in this case. FIG. 6 illustrates several sample operations that may be performed in conjunction with conditionally providing anodal cardiac stimulation.

As represented by block 602, in some embodiments a condition for providing anodal cardiac stimulation may be determined based on one or more sensed signals. For example, the apparatus 200 (e.g., the sense circuit 204) may monitor signals that are indicative of a heart failure condition of a patient or an ischemia condition of a patient. For the case of heart failure, such signals may relate to, for example, a cardiac impedance measurement, an evoked response, QRS timing, or some other physiological characteristic.

As represented by blocks 604 and 606, a condition analyzer 206 may monitor one or more conditions of the patient (e.g., by analyzing the sensed signals over time) and determine whether to provide anodal stimulation or cathodal stimulation. For example, if the heart failure or ischemia condition of a patient has not digressed beyond a certain degree, the condition analyzer 206 may indicate that cathodal stimulation should be used. In contrast, if a patient is suffering from worsening heart failure or worsening ischemia, the condition analyzer 206 may indicate that anodal stimulation should be used. In this latter case, if the heart failure or ischemia condition improves at a later point in time, the condition analyzer 206 may then indicate that cathodal stimulation should be used.

As represented by block 608, the stimulation controller 210 may then provide the designated form of cardiac stimulation whenever the need for cardiac stimulation is indication. That is, during normal sensing and stimulation operations, if it is determined that a certain intrinsic event (e.g., P-wave or R-wave) was not detected, the stimulation controller 210 may provide anodal stimulation if the associated condition is met or the conditions are met at block 606.

In some embodiments, different conditions may be used to control the cardiac stimulation provided by the apparatus 200. For example, a condition for providing anodal stimulation may relate to the time of day (e.g., only provide anodal stimulation at defined times such as at night), an environmental condition, patient activity level, or some other suitable factor. In some cases, the amount of cardiac stimulation provided by the apparatus 200 may be restricted in some manner. For example, the apparatus 200 may be limited to provide cardiac stimulation for only a certain number of hours per day (e.g., 10% of the day.)

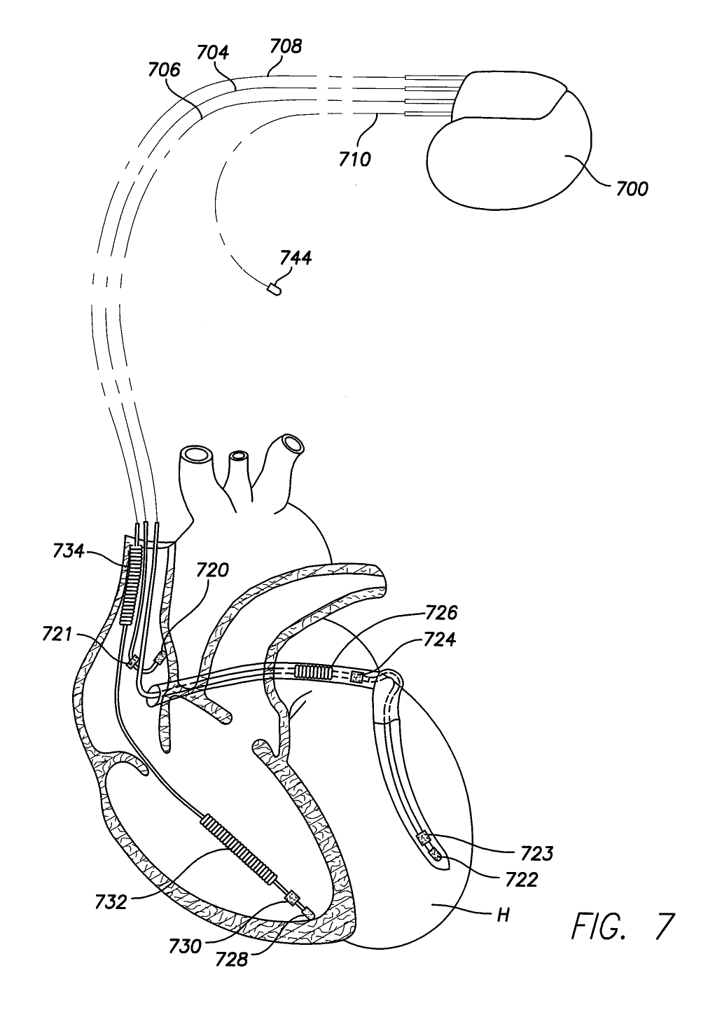

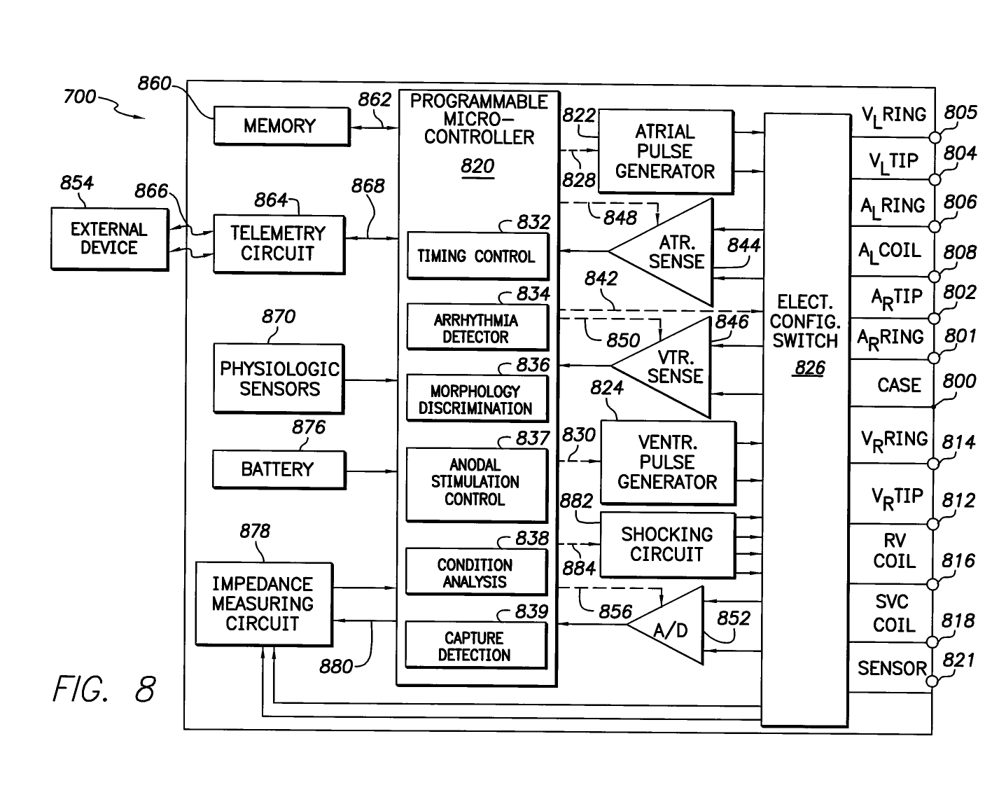

Referring now to FIGS. 7 and 8, an example of an implantable cardiac device (e.g., a stimulation device such as an implantable cardioverter defibrillator, a pacemaker, etc.) that may be implemented in accordance with the teachings herein will be described. It is to be appreciated and understood that other cardiac devices, including those that are not necessarily implantable, may be used and that the description below is given, in its specific context, to assist the reader in understanding, with more clarity, the embodiments described herein.

FIG. 7 shows an exemplary implantable cardiac device 700 in electrical communication with a patient's heart H by way of three leads 704, 706, and 708, suitable for delivering multi-chamber stimulation and shock therapy. To sense atrial cardiac signals and to provide right atrial chamber stimulation therapy, the device 700 is coupled to an implantable right atrial lead 704 having, for example, an atrial tip electrode 720, which typically is implanted in the patient's right atrial appendage or septum. FIG. 7 also shows the right atrial lead 704 as having an optional atrial ring electrode 721.

To sense left atrial and ventricular cardiac signals and to provide left chamber pacing therapy, the device 700 is coupled to a coronary sinus lead 706 designed for placement in the coronary sinus region via the coronary sinus for positioning one or more electrodes adjacent to the left ventricle, one or more electrodes adjacent to the left atrium, or both. As used herein, the phrase “coronary sinus region” refers to the vasculature of the left ventricle, including any portion of the coronary sinus, the great cardiac vein, the left marginal vein, the left posterior ventricular vein, the middle cardiac vein, the small cardiac vein or any other cardiac vein accessible by the coronary sinus.

Accordingly, an exemplary coronary sinus lead 706 is designed to receive atrial and ventricular cardiac signals and to deliver left ventricular pacing therapy using, for example, a left ventricular tip electrode 722 and, optionally, a left ventricular ring electrode 723; provide left atrial pacing therapy using, for example, a left atrial ring electrode 724; and provide shocking therapy using, for example, a left atrial coil electrode 726 (or other electrode capable of delivering a shock). For a more detailed description of a coronary sinus lead, the reader is directed to U.S. Pat. No. 5,466,254, “Coronary Sinus Lead with Atrial Sensing Capability”, which is incorporated herein by reference.

The device 700 is also shown in electrical communication with the patient's heart H by way of an implantable right ventricular lead 708 having, in this implementation, a right ventricular tip electrode 728, a right ventricular ring electrode 730, a right ventricular (RV) coil electrode 732 (or other electrode capable of delivering a shock), and a superior vena cava (SVC) coil electrode 734 (or other electrode capable of delivering a shock). Typically, the right ventricular lead 708 is transvenously inserted into the heart H to place the right ventricular tip electrode 728 in the right ventricular apex so that the RV coil electrode 732 will be positioned in the right ventricle and the SVC coil electrode 734 will be positioned in the superior vena cava. Accordingly, the right ventricular lead 708 is capable of sensing or receiving cardiac signals, and delivering stimulation in the form of pacing and shock therapy to the right ventricle.

The device 700 is also shown in electrical communication with a lead 710 including one or more components 744 such as a physiologic sensor. The component 744 may be positioned in, near or remote from the heart.

It should be appreciated that the device 700 may connect to leads other than those specifically shown. In addition, the leads connected to the device 700 may include components other than those specifically shown. For example, a lead may include other types of electrodes, sensors or devices that serve to otherwise interact with a patient or the surroundings.

FIG. 8 depicts an exemplary, simplified block diagram illustrating sample components of the device 700. The device 700 may be adapted to treat both fast and slow arrhythmias with stimulation therapy, including cardioversion, defibrillation, and pacing stimulation. While a particular multi-chamber device is shown, it is to be appreciated and understood that this is done for illustration purposes. Thus, the techniques and methods described below can be implemented in connection with any suitably configured or configurable device. Accordingly, one of skill in the art could readily duplicate, eliminate, or disable the appropriate circuitry in any desired combination to provide a device capable of treating the appropriate chamber(s) with, for example, cardioversion, defibrillation, and pacing stimulation.

Housing 800 for the device 700 is often referred to as the “can”, “case” or “case electrode”, and may be programmably selected to act as the return electrode for all “unipolar” modes. The housing 800 may further be used as a return electrode alone or in combination with one or more of the coil electrodes 726, 732 and 734 for shocking purposes. Housing 800 further includes a connector (not shown) having a plurality of terminals 801, 802, 804, 805, 806, 808, 812, 814, 816 and 818 (shown schematically and, for convenience, the names of the electrodes to which they are connected are shown next to the terminals). The connector may be configured to include various other terminals (e.g., terminal 821 coupled to a sensor or some other component) depending on the requirements of a given application.

To achieve right atrial sensing and pacing, the connector includes, for example, a right atrial tip terminal (A_{R} TIP) 802 adapted for connection to the right atrial tip electrode 720. A right atrial ring terminal (A_{R} RING) 801 may also be included and adapted for connection to the right atrial ring electrode 721. To achieve left chamber sensing, pacing, and shocking, the connector includes, for example, a left ventricular tip terminal (V_{L} TIP) 804, a left ventricular ring terminal (V_{L} RING) 805, a left atrial ring terminal (A_{L} RING) 806, and a left atrial shocking terminal (A_{L} COIL) 808, which are adapted for connection to the left ventricular tip electrode 722, the left ventricular ring electrode 723, the left atrial ring electrode 724, and the left atrial coil electrode 726, respectively.

To support right chamber sensing, pacing, and shocking, the connector further includes a right ventricular tip terminal (V_{R} TIP) 812, a right ventricular ring terminal (V_{R} RING) 814, a right ventricular shocking terminal (RV COIL) 816, and a superior vena cava shocking terminal (SVC COIL) 818, which are adapted for connection to the right ventricular tip electrode 728, the right ventricular ring electrode 730, the RV coil electrode 732, and the SVC coil electrode 734, respectively.

At the core of the device 700 is a programmable microcontroller 820 that controls the various modes of stimulation therapy. As is well known in the art, microcontroller 820 typically includes a microprocessor, or equivalent control circuitry, designed specifically for controlling the delivery of stimulation therapy, and may further include memory such as RAM, ROM and flash memory, logic and timing circuitry, state machine circuitry, and I/O circuitry. Typically, microcontroller 820 includes the ability to process or monitor input signals (data or information) as controlled by a program code stored in a designated block of memory. The type of microcontroller is not critical to the described implementations. Rather, any suitable microcontroller 820 may be used that carries out the functions described herein. The use of microprocessor-based control circuits for performing timing and data analysis functions are well known in the art.

Representative types of control circuitry that may be used in connection with the described embodiments can include the microprocessor-based control system of U.S. Pat. No. 4,940,052, the state-machine of U.S. Pat. Nos. 4,712,555 and 4,944,298, all of which are incorporated by reference herein. For a more detailed description of the various timing intervals that may be used within the device and their inter-relationship, see U.S. Pat. No. 4,788,980, also incorporated herein by reference.

FIG. 8 also shows an atrial pulse generator 822 and a ventricular pulse generator 824 that generate pacing stimulation pulses for delivery by the right atrial lead 704, the coronary sinus lead 706, the right ventricular lead 708, or some combination of these leads via an electrode configuration switch 826. It is understood that in order to provide stimulation therapy in each of the four chambers of the heart, the atrial and ventricular pulse generators 822 and 824 may include dedicated, independent pulse generators, multiplexed pulse generators, or shared pulse generators. The pulse generators 822 and 824 are controlled by the microcontroller 820 via appropriate control signals 828 and 830, respectively, to trigger or inhibit the stimulation pulses.

Microcontroller 820 further includes timing control circuitry 832 to control the timing of the stimulation pulses (e.g., pacing rate, atrio-ventricular (A-V) delay, atrial interconduction (A-A) delay, or ventricular interconduction (V-V) delay, etc.) or other operations, as well as to keep track of the timing of refractory periods, blanking intervals, noise detection windows, evoked response windows, alert intervals, marker channel timing, etc., as known in the art.

Microcontroller 820 further includes an arrhythmia detector 834. The arrhythmia detector 834 may be utilized by the device 700 for determining desirable times to administer various therapies. The arrhythmia detector 834 may be implemented, for example, in hardware as part of the microcontroller 820, or as software/firmware instructions programmed into the device 700 and executed on the microcontroller 820 during certain modes of operation.

Microcontroller 820 also includes a morphology discrimination module 836, a capture detection module 839 and an auto sensing module (not shown). These modules may be used to implement various exemplary recognition algorithms or methods. The aforementioned components may be implemented, for example, in hardware as part of the microcontroller 820, or as software/firmware instructions programmed into the device 700 and executed on the microcontroller 820 during certain modes of operation.

The electrode configuration switch 826 includes a plurality of switches for connecting the desired terminals (e.g., that are connected to electrodes, coils, sensors, etc.) to the appropriate I/O circuits, thereby providing complete terminal and, hence, electrode programmability. Accordingly, switch 826, in response to a control signal 842 from the microcontroller 820, may be used to determine the polarity of the stimulation pulses (e.g., unipolar, bipolar, combipolar, etc.) by selectively closing the appropriate combination of switches (not shown) as is known in the art.

Atrial sensing circuits 844 and ventricular sensing circuits 846 may also be selectively coupled to the right atrial lead 704, coronary sinus lead 706, and the right ventricular lead 708, through the switch 826 for detecting the presence of cardiac activity in each of the four chambers of the heart. Accordingly, the atrial and ventricular sensing circuits 844, 846 may include dedicated sense amplifiers, multiplexed amplifiers, or shared amplifiers. Switch 826 determines the “sensing polarity” of the cardiac signal by selectively closing the appropriate switches, as is also known in the art. In this way, the clinician may program the sensing polarity independent of the stimulation polarity. The sensing circuits (e.g., circuits 844, 846) are optionally capable of obtaining information indicative of tissue capture.

Each sensing circuit 844, 846 preferably employs one or more low power, precision amplifiers with programmable gain, automatic gain control, bandpass filtering, a threshold detection circuit, or some combination of these components, to selectively sense the cardiac signal of interest. The automatic gain control enables the device 700 to deal effectively with the difficult problem of sensing the low amplitude signal characteristics of atrial or ventricular fibrillation.

The outputs of the atrial and ventricular sensing circuits 844, 846 are connected to the microcontroller 820, which, in turn, is able to trigger or inhibit the atrial and ventricular pulse generators 822, 824, respectively, in a demand fashion in response to the absence or presence of cardiac activity in the appropriate chambers of the heart. Furthermore, as described herein, the microcontroller 820 is also capable of analyzing information output from the sensing circuits 844, 846, a data acquisition system 852, or both. This information may be used to determine or detect whether and to what degree tissue capture has occurred and to program a pulse, or pulses, in response to such determinations. The sensing circuits 844, 846, in turn, receive control signals over signal lines 848, 850, respectively, from the microcontroller 820 for purposes of controlling the gain, threshold, polarization charge removal circuitry (not shown), and the timing of any blocking circuitry (not shown) coupled to the inputs of the sensing circuits 844, 846 as is known in the art.

For arrhythmia detection, the device 700 utilizes the atrial and ventricular sensing circuits 844, 846 to sense cardiac signals to determine whether a rhythm is physiologic or pathologic. It should be appreciated that other components may be used to detect arrhythmia depending on the system objectives. In reference to arrhythmias, as used herein, “sensing” is reserved for the noting of an electrical signal or obtaining data (information), and “detection” is the processing (analysis) of these sensed signals and noting the presence of an arrhythmia.

Timing intervals between sensed events (e.g., P-waves, R-waves, and depolarization signals associated with fibrillation) may be classified by the arrhythmia detector 834 of the microcontroller 820 by comparing them to a predefined rate zone limit (e.g., bradycardia, normal, low rate VT, high rate VT, and fibrillation rate zones) and various other characteristics (e.g., sudden onset, stability, physiologic sensors, and morphology, etc.) in order to determine the type of remedial therapy that is needed (e.g., bradycardia pacing, anti-tachycardia pacing, cardioversion shocks or defibrillation shocks, collectively referred to as “tiered therapy”). Similar rules may be applied to the atrial channel to determine if there is an atrial tachyarrhythmia or atrial fibrillation with appropriate classification and intervention.

Cardiac signals or other signals may be applied to inputs of an analog-to-digital (A/D) data acquisition system 852. The data acquisition system 852 is configured (e.g., via signal line 856) to acquire intracardiac electrogram (“IEGM”) signals or other signals, convert the raw analog data into a digital signal, and store the digital signals for later processing, for telemetric transmission to an external device 854, or both. For example, the data acquisition system 852 may be coupled to the right atrial lead 704, the coronary sinus lead 706, the right ventricular lead 708 and other leads through the switch 826 to sample cardiac signals across any pair of desired electrodes.

The data acquisition system 852 also may be coupled to receive signals from other input devices. For example, the data acquisition system 852 may sample signals from a physiologic sensor 870 or other components shown in FIG. 8 (connections not shown).

In some aspects, the data acquisition system 852 may be employed to record an IEGM signal during a window following delivery of a pacing pulse to enable the capture detection module 839 to detect capture of a desired chamber of the heart in response to the applied pacing stimulus. As discussed above, capture occurs when an electrical stimulus applied to the heart is of sufficient energy to depolarize the cardiac tissue, thereby causing the heart muscle to contract. The microcontroller 820 enables capture detection when a pulse generator (e.g., pulse generator 822 or 824) generates a stimulation pulse. During the stimulation pulse, the inputs to a sense circuit (not shown) of the data acquisition system 852 may be shorted (e.g., a blanking period may be defined at this time). After the stimulation pulse, the microcontroller 820 may start a detection window (e.g., on the order of 64 mS), using the timing control circuitry 832 of the microcontroller 820. During this window, the data acquisition system 852 (e.g., in response to a control signal 856) samples the IEGM signal that occurs during the capture detection window and stores the IEGM in the memory 860. Thereafter, the microcontroller 820 processes the IEGM to obtain a measurement related to capture. For example, microcontroller 820 may integrate the stored IEGM with respect to a baseline established during the blanking period. If the resulting integral is greater than a threshold determined by a threshold circuit (not shown) of the capture detection module 839, capture is deemed to have occurred. The threshold may be set manually through programming or automatically by the threshold circuit to eliminate false positives. Capture detection may be invoked on a beat-by-beat basis, on a sampled basis (e.g., every Nth beat), or in some other manner.

Capture detection also may be employed during a capture threshold search. Such a capture threshold search may be performed, for example, once a day during at least the acute phase (e.g., the first 30 days) and less frequently thereafter. As mentioned above, a capture threshold search may begin at a desired energy level starting point and the energy level is adjusted until capture is lost or achieved.

The microcontroller 820 is further coupled to a memory 860 by a suitable data/address bus 862, wherein the programmable operating parameters used by the microcontroller 820 are stored and modified, as required, in order to customize the operation of the device 700 to suit the needs of a particular patient. Such operating parameters define, for example, pacing pulse amplitude, pulse duration, electrode polarity, rate, sensitivity, automatic features, arrhythmia detection criteria, and the amplitude, waveshape and vector of each shocking pulse to be delivered to the patient's heart H within each respective tier of therapy. One feature of the described embodiments is the ability to sense and store a relatively large amount of data (e.g., from the data acquisition system 852), which data may then be used for subsequent analysis to guide the programming of the device 700.

Advantageously, the operating parameters of the implantable device 700 may be non-invasively programmed into the memory 860 through a telemetry circuit 864 in telemetric communication via communication link 866 with the external device 854, such as a programmer, transtelephonic transceiver, a diagnostic system analyzer or some other device. The microcontroller 820 activates the telemetry circuit 864 with a control signal (e.g., via bus 868). The telemetry circuit 864 advantageously allows intracardiac electrograms and status information relating to the operation of the device 700 (as contained in the microcontroller 820 or memory 860) to be sent to the external device 854 through an established communication link 866.

The device 700 can further include one or more physiologic sensors 870. In some embodiments the device 700 may include a “rate-responsive” sensor that may provide, for example, information to aid in adjustment of pacing stimulation rate according to the exercise state of the patient. One or more physiologic sensors 870 (e.g., a pressure sensor) may further be used to detect changes in cardiac output, changes in the physiological condition of the heart, or diurnal changes in activity (e.g., detecting sleep and wake states). Accordingly, the microcontroller 820 responds by adjusting the various pacing parameters (such as rate, A-V Delay, V-V Delay, etc.) at which the atrial and ventricular pulse generators 822 and 824 generate stimulation pulses.

While shown as being included within the device 700, it is to be understood that a physiologic sensor 870 may also be external to the device 700, yet still be implanted within or carried by the patient. Examples of physiologic sensors that may be implemented in conjunction with the device 700 include sensors that sense respiration rate, pH of blood, ventricular gradient, oxygen saturation, blood pressure and so forth. Another sensor that may be used is one that detects activity variance, wherein an activity sensor is monitored diurnally to detect the low variance in the measurement corresponding to the sleep state. For a more detailed description of an activity variance sensor, the reader is directed to U.S. Pat. No. 5,476,483 (Bornzin et al.), which patent is hereby incorporated by reference.

The one or more physiologic sensors 870 may optionally include one or more of components to help detect movement (via, e.g., a position sensor or an accelerometer) and minute ventilation (via an MV sensor) in the patient. Signals generated by the position sensor and MV sensor may be passed to the microcontroller 820 for analysis in determining whether to adjust the pacing rate, etc. The microcontroller 820 may thus monitor the signals for indications of the patient's position and activity status, such as whether the patient is climbing up stairs or descending down stairs or whether the patient is sitting up after lying down.

The device 700 additionally includes a battery 876 that provides operating power to all of the circuits shown in FIG. 8. For a device 700 which employs shocking therapy, the battery 876 is capable of operating at low current drains (e.g., preferably less than 10 μA) for long periods of time, and is capable of providing high-current pulses (for capacitor charging) when the patient requires a shock pulse (e.g., preferably, in excess of 2 A, at voltages above 200 V, for periods of 10 seconds or more). The battery 876 also desirably has a predictable discharge characteristic so that elective replacement time can be detected. Accordingly, the device 700 preferably employs lithium (e.g., lithium/silver vanadium) or some other suitable battery technology.

The device 700 can further include magnet detection circuitry (not shown), coupled to the microcontroller 820, to detect when a magnet is placed over the device 700. A magnet may be used by a clinician to perform various test functions of the device 700 and to signal the microcontroller 820 that the external device 854 is in place to receive data from or transmit data to the microcontroller 820 through the telemetry circuit 864.

The device 700 further includes an impedance measuring circuit 878 that is enabled by the microcontroller 820 via a control signal 880. The known uses for an impedance measuring circuit 878 include, but are not limited to, lead impedance surveillance during the acute and chronic phases for proper performance, lead positioning or dislodgement; detecting operable electrodes and automatically switching to an operable pair if dislodgement occurs; measuring respiration or minute ventilation; measuring thoracic impedance for determining shock thresholds; detecting when the device 700 has been implanted; measuring stroke volume; and detecting the opening of heart valves, etc. The impedance measuring circuit 878 is advantageously coupled to the switch 826 so that any desired electrode may be used. In some embodiments the impedance measuring circuit 878 may be used for detecting capture, detecting a condition of a patient, and determining a pacing current as discussed herein.

In the case where the device 700 is intended to operate as an implantable cardioverter/defibrillator (ICD) device, it detects the occurrence of an arrhythmia, and automatically applies an appropriate therapy to the heart aimed at terminating the detected arrhythmia. To this end, the microcontroller 820 further controls a shocking circuit 882 by way of a control signal 884. The shocking circuit 882 generates shocking pulses of low (e.g., up to 0.5 J), moderate (e.g., 0.5 J to 10 J), or high energy (e.g., 11 J to 40 J), as controlled by the microcontroller 820. Such shocking pulses are applied to the patient's heart H through, for example, two shocking electrodes and as shown in this embodiment, selected from the left atrial coil electrode 726, the RV coil electrode 732 and the SVC coil electrode 734. As noted above, the housing 800 may act as an active electrode in combination with the RV coil electrode 732, as part of a split electrical vector using the SVC coil electrode 734 or the left atrial coil electrode 726 (i.e., using the RV electrode as a common electrode), or in some other arrangement.

Cardioversion level shocks are generally considered to be of low to moderate energy level (so as to minimize pain felt by the patient), be synchronized with an R-wave, pertain to the treatment of tachycardia, or some combination of the above. Defibrillation shocks are generally of moderate to high energy level (i.e., corresponding to thresholds in the range of 5 J to 40 J), delivered asynchronously (since R-waves may be too disorganized), and pertaining to the treatment of fibrillation. Accordingly, the microcontroller 820 is capable of controlling the synchronous or asynchronous delivery of the shocking pulses.

As mentioned above, the device 700 may include several components that provide the anodal stimulation related functionality as taught herein. For example, one or more of the switch 826, the sense circuits 844, 846 or the data acquisition system 852 may acquire cardiac signals that are used to determine whether capture has been achieved as discussed above, with reference to FIGS. 1, 3, and 4. Also, one or more of the switch 826, the ventricular pulse generator 824, or the atrial pulse generator 822 may be used to provide anodal stimulation as discussed above, with reference to FIGS. 1 and 3-6. The data described above may be stored in the data memory 860. The components of FIG. 8 may thus provide functionality described above with reference to FIG. 2. For example, the switch 202 of FIG. 2 may correspond to the switch 826 of FIG. 8. In addition, the sense circuit 204 may correspond to one or more of the atrial sense circuit 844, the ventricular sense circuit 846, or the data acquisition system 852.

The microcontroller 820 (e.g., a processor providing signal processing functionality) also may implement or support at least a portion of the anodal stimulation related functionality discussed herein. For example, a capture detection component 839 (e.g., corresponding to the capture threshold detector 208) may perform capture threshold detection operations as described above with reference to FIGS. 1, 3, and 4. An anodal stimulation control component 837 (e.g., corresponding to the stimulation controller 210 and the stimulation scheduler 212) may perform cardiac stimulation operations as described above with reference to FIGS. 1 and 3-6. A condition analysis component 838 (e.g., corresponding to the condition analyzer 206) may perform condition analysis operations as described above with reference to FIGS. 1 and 6.

It should be appreciated that various modifications may be incorporated into the disclosed embodiments based on the teachings herein. For example, the structure and functionality taught herein may be incorporated into types of devices other than the specific types of devices described above. In addition, based on the teachings herein various techniques may be used to detect anodal capture and provide anodal stimulation/pacing.

It should be appreciated from the above that the various structures and functions described herein may be incorporated into a variety of apparatuses (e.g., a stimulation device, a lead, a monitoring device, etc.) and implemented in a variety of ways. Different embodiments of such an apparatus may include a variety of hardware and software processing components. In some embodiments, hardware components such as processors, controllers, state machines, logic, or some combination of these components, may be used to implement the described components or circuits.

In some embodiments, code including instructions (e.g., software, firmware, middleware, etc.) may be executed on one or more processing devices to implement one or more of the described functions or components. The code and associated components (e.g., data structures and other components used by the code or used to execute the code) may be stored in an appropriate data memory that is readable by a processing device (e.g., commonly referred to as a computer-readable medium).

Moreover, some of the operations described herein may be performed by a device that is located externally with respect to the body of the patient. For example, an implanted device may send raw data or processed data to an external device that then performs the necessary processing.

The components and functions described herein may be connected or coupled in many different ways. The manner in which this is done may depend, in part, on whether and how the components are separated from the other components. In some embodiments some of the connections or couplings represented by the lead lines in the drawings may be in an integrated circuit, on a circuit board or implemented as discrete wires or in other ways.

Moreover, the recited order of the blocks in the processes disclosed herein is simply an example of a suitable approach. Thus, operations associated with such blocks may be rearranged while remaining within the scope of the present disclosure. Similarly, the accompanying method claims present operations in a sample order, and are not necessarily limited to the specific order presented.

Also, it should be understood that any reference to elements herein using a designation such as “first,” “second,” and so forth does not generally limit the quantity or order of those elements. Rather, these designations may be used herein as a convenient method of distinguishing between two or more different elements or instances of an element. Thus, a reference to first and second elements does not mean that only two elements may be employed there or that the first element must precede the second element in some manner. Also, unless stated otherwise a set of elements may comprise one or more elements.

While certain embodiments have been described above in detail and shown in the accompanying drawings, it is to be understood that such embodiments are merely illustrative of and not restrictive of the teachings herein. In particular, it should be recognized that the teachings herein apply to a wide variety of apparatuses and methods. It will thus be recognized that various modifications may be made to the illustrated embodiments or other embodiments, without departing from the broad scope thereof. In view of the above it will be understood that the teachings herein are intended to cover any changes, adaptations or modifications which are within the scope of the disclosure.

What is claimed is:

1. A method of cardiac stimulation, comprising:
sensing cardiac signals;
determining a cardiac condition based on the sensed cardiac signals; and
determining whether to provide anodal cardiac stimulation or cathodal cardiac stimulation based on the determined cardiac condition.

2. The method of claim 1, further comprising providing the anodal cardiac stimulation if the determined cardiac condition relates to a worsening heart failure condition.

3. The method of claim 1, further comprising providing the anodal cardiac stimulation if the determined cardiac condition relates to a worsening ischemia condition.

4. The method of claim 1, wherein the sensed signals indicate cardiac impedance.

5. The method of claim 1, wherein the sensed signals indicate at least one evoked response.

6. The method of claim 1, further comprising providing the anodal cardiac stimulation at defined times.

7. The method of claim 1, further comprising providing the anodal cardiac stimulation or the cathodal cardiac stimulation according to a defined anodal/cathodal stimulation ratio.

8. The method of claim 1, further comprising providing anodal cardiac stimulation that effectuates anodal cardiac capture at a plurality of anodal electrodes by:
determining a first current level associated with an anodal cardiac capture threshold for a first anodal electrode;
determining a second current level associated with an anodal cardiac capture threshold for a second anodal electrode; and
defining, based on the first and second current levels, an anodal cardiac stimulation energy level sufficient to provide anodal cardiac capture at the first and second anodal electrodes.

9. An implantable cardiac stimulation device comprising:
a cardiac sensor configured to sense cardiac signals;
a cardiac condition analyzer configured to determine a cardiac condition based on the sensed cardiac signals; and
a cardiac stimulation controller configured to determine whether to provide anodal cardiac stimulation or cathodal cardiac stimulation based on the determined cardiac condition.

10. A method of cardiac stimulation, comprising:
defining anodal stimulation times and cathodal stimulation times according to a defined anodal/cathodal stimulation ratio;
generating anodal cardiac stimulation signals according to the defined anodal stimulation times; and
generating cathodal cardiac stimulation signals according to the defined cathodal stimulation times.

11. The method of claim 10, wherein the defined anodal stimulation times and the defined cathodal stimulation times are mutually exclusive.

12. The method of claim 10, wherein the defined anodal/cathodal stimulation ratio defines a first percentage of time for anodal cardiac stimulation and a second percentage of time for cathodal cardiac stimulation.

13. The method of claim 10, wherein the defined anodal/cathodal stimulation ratio defines a first quantity of anodal cardiac stimulation pulses and a second quantity of cathodal cardiac stimulation pulses.

14. The method of claim 10, further comprising determining whether to generate the anodal cardiac stimulation signals based on whether a heart failure or ischemia condition is worsening.

15. An implantable cardiac stimulation device, comprising:
a cardiac stimulation scheduler configure to define anodal stimulation times and cathodal stimulation times according to a defined anodal/cathodal stimulation ratio; and
a cardiac stimulation controller configured to generate anodal cardiac stimulation signals according to the defined anodal stimulation times and generate cathodal cardiac stimulation signals according to the defined cathodal stimulation times.

16. A method of cardiac stimulation, comprising:
configuring at least one implantable electrode as an anode;
adjusting cardiac stimulation signals provided to the at least one implantable electrode and monitoring evoked response signals that result from the cardiac stimulation signals to identify an anodal cardiac capture threshold;
determining a stimulation energy level associated with the identified anodal cardiac capture threshold; and
generating anodal cardiac stimulation signals in accordance with the determined stimulation energy level.

17. The method of claim 16, further comprising:
configuring at least one other implantable electrode as an anode;
adjusting other cardiac stimulation signals provided to the at least one other implantable electrode and monitor other evoked response signals that result from the other cardiac stimulation signals to identify another anodal cardiac capture threshold;
determining another stimulation energy level associated with the identified another anodal cardiac capture threshold; and
generating the anodal cardiac stimulation signals in accordance with a lowest one of the determined stimulation energy level and the determined another stimulation energy level.

18. The method of claim 16, further comprising generating anodal cardiac stimulation signals that effectuate anodal cardiac capture at a plurality of anodal electrodes by:
determining a first current level associated with an anodal cardiac capture threshold for a first anodal electrode;
determining a second current level associated with an anodal cardiac capture threshold for a second anodal electrode; and
defining, based on the first and second current levels, an anodal cardiac stimulation energy level sufficient to provide anodal cardiac capture at the first and second anodal electrodes.

19. The method of claim 16, further comprising distinguishing anodal cardiac capture and cathodal cardiac capture based on a time difference between a first evoked response associated with the anodal cardiac capture and a second evoked response associated with the cathodal cardiac capture.

20. The method of claim 16, further comprising selecting the least one other implantable electrode based on relative sizes of a plurality of implantable electrodes.

21. An implantable cardiac stimulation device, comprising:
a switch configured to configure at least one implantable electrode as an anode;
a cardiac capture threshold detector configured to adjust cardiac stimulation signals provided to the at least one implantable electrode and monitor evoked response signals that result from the cardiac stimulation signals to identify an anodal cardiac capture threshold; and
a cardiac stimulation controller configured to determine a stimulation energy level associated with the identified anodal cardiac capture threshold, and further configured to generate anodal cardiac stimulation signals in accordance with the determined stimulation energy level.
